(19) United States
(12) Patent Application Publication
(10) Pub. No.: US 2023/0277540 A1
(43) Pub. Date: Sep. 7, 2023

(54) Treatment of Dilated Cardiomyopathies

(71) Applicants: Genethon, Evry-Courcouronnes (FR); Universite d'Evry Val d'Essonne, Evry (FR); INSERM (INSTITUT NATIONAL DE LA SANTÉ ET DE LA RECHERCHE MÉDICALE), PARIS (FR)

(72) Inventors: Isabelle Richard, Corbeil-Essonnes (FR); Ariane Biquand, Maisons Alfort (FR)

(73) Assignees: Genethon, Evry-Courcouronnes (FR); Universite d'Evry Val d'Essonne, Evry (FR); Institut National de la Santé et de la Recherche Médicale, Paris (FR)

(21) Appl. No.: 18/009,034

(22) PCT Filed: Jun. 9, 2021
(86) PCT No.: PCT/EP2021/065432

§ 371 Date: Dec. 8, 2022

(30) Foreign Application Priority Data
Jun. 9, 2020 (EP) 20305622.1

(51) Int. Cl. A61K 31/519; A61K 31/4439; A61K 31/7088; A61P 9/00

(52) U.S. Cl. CPC A61K 31/519; A61K 31/4439; A61K 31/7088; A61P 9/00

(57) ABSTRACT

The invention relates to the treatment of dilated cardiomyopathies, in particular genetic dilated cardiomyopathies, using non-expressible inhibitors of the WNT pathway or TGF-β pathway, alone or in combination.

### FIELD OF THE INVENTION

The invention relates to the treatment of dilated cardiomyopathies, in particular genetic dilated cardiomyopathies, using non-expressible inhibitors of the WNT pathway or TGF-β pathway, alone or in combination.

### BACKGROUND OF THE INVENTION

Cardiomyopathy and heart failure remain, despite management, one of the major causes of morbidity and mortality worldwide. Dilated cardiomyopathies (DCM or CMD) are characterized by hypokinesis of the myocardium and dilatation of the cardiac cavities. The cardiac remodelling that takes place during dilated cardiomyopathies consists of damage to the cardiomyocytes associated with the presence of fibrosis, which are inseparable from each other. The damage to the cardiomyocytes involves a decrease in their contractile capacity and a change in their structure, which leads to apoptosis and to the expansion of fibrosis, which replaces the necrotic cardiomyocytes. The proliferation of fibroblasts prevents compensatory hypertrophy of the cardiomyocytes. These manifestations will clinically translate into a decrease in cardiac function. This serious complication can be a cause of death.

Causes include in particular genetics, and a variety of toxic, metabolic or infectious agents. Coronary artery disease and high blood pressure may play a role, but are not the primary cause. In many cases, the cause remains unclear. The exact mechanism of cardiomyocyte involvement depends on the etiology of the disease. In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RYR2, SERCA2a (ATP2A2), phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength. In Duchenne muscular dystrophy (DMD), a muscle disease due to mutation in the dystrophin gene, a dilated cardiomyopathy is clinically revealed around the age of 15 years and affects almost all patients after the age of 20 years. In the case of Becker muscular dystrophy (BMD), an allelic form of DMD, cardiac damage develops at the age of 20 years, and 70% of patients are affected after the age of 35. DCM due to titin, a giant protein of the sarcomere is implicated in 1/250 cases of heart failure (Burke et al., JCI Insight. 2016; 1(6): e86898. doi:10.1172/jci.insight.86898).

The drugs currently available for the treatment of dilated cardiomyopathies will improve the symptoms but not treat the causative mechanisms of the disease. The treatments prescribed are those for heart failure, accompanied by hygienic and dietetic measures such as reducing alcohol consumption, reducing water and salt intake and moderate and regular physical exercise. Among pharmaceutical treatments, angiotensin II converting enzyme inhibitors (ACE inhibitors) prevent the production of angiotensin II in order to decrease vasoconstriction and blood pressure. Diuretic drugs remove excess salt and water from the body by inhibiting renal sodium reabsorption. β-blockers, or β-adrenergic receptor antagonists, block the effects of adrenergic system mediators stimulated during dilated cardiomyopathies and decrease heart rate. Mineral-corticoid receptor antagonists block the binding of aldosterone and lower blood pressure. When rhythm disturbances are severe, anti-arrhythmic drugs such as amiodarone are prescribed. Implantation of a pacemaker and/or automatic defibrillator may also be considered. In the most severe cases, patients may benefit from a heart transplant (Ponikowski et al., European Heart Journal, 2016, 37, 2129-2200).

These approaches are therefore also valid for the management of dilated cardiomyopathies in cases of DMD and titinopathies. There is currently no curative treatment for these pathologies. Corticosteroid treatment, frequently prescribed in DMD, allows an improvement of the muscular phenotype in the medium term thanks to a reduction in inflammation, but its action on the cardiac phenotype is subject to debate. The management of DMD related to dystrophin and titin requires an annual and systematic cardiac check-up (electrocardiogram and ultrasound). In particular, Perindopril, an angiotensin-converting enzyme inhibitor, has been shown to reduce mortality in DMD patients when taken as a preventive treatment from childhood onwards (Duboc et al., Journal of the American College of Cardiology, 2005, 45, 855-857). Molecules being tested in the treatment of cardiac impairment in DMD are mainly molecules already used in the treatment of heart failure. Other therapies are aimed at treating muscle and heart damage by reducing fibrosis. This is the case for Pamrevlumab (Phase II trial NCT02606136), a monoclonal antibody directed against connective tissue growth factor, and Tamoxifen (Phase I trial NCT02835079 and Phase III trial NCT03354039), an anti-estrogen.

There is therefore a medical need to develop new therapeutic strategies for dilated cardiomyopathies, in particular genetic dilated cardiomyopathies.

The WNT (or Wnt) pathway orchestrates various biological processes, such as cell proliferation, differentiation, organogenesis, tissue regeneration and tumorigenesis. Classically, Wnt signaling is divided into β-catenin dependent (canonical, Wnt/β-catenin pathway) and β-catenin independent (noncanonical, Wnt/planar cell polarity (PCO) and calcium pathway). All three are activated by the binding of different secreted glycoproteins (Wnt ligands) to the frizzled (FZD) receptor family, to transduce a signaling cascade from the dishevelled protein (DVL) in the cell. The Wnt pathway is modulated by endogenous antagonists such as dickkopf, Wnt inhibitory signaling protein, secreted frizzled related protein and cerberus. The secretion of WNT proteins mainly depends on acylation by porcupine (PORCN). β-catenin is a crucial signaling transducer in Wnt signaling. The β-catenin protein destruction complex composed of adenomatous polyposis coli (APC), casein kinase 1 (CK1) and glycogen synthase kinase 3α/β (GSK-3α/β) and axin tightly controls 0-catenin via phosphorylation-mediated proteolysis. The poly-ADP-ribosylating enzyme tankyrase interacts with and degrades axin via ubiquitin-mediated proteasomal degradation. In the absence of a ligand, the β-catenin accumulated in the cytoplasm is degraded by the destruction complex. Upon binding of one of the secreted Wnt proteins to its frizzled receptor (FZD) and lipoprotein co-receptor (LRP5/6), the cytoplasmic β-catenin is stabilized and translocated into the nucleus where it interacts with the transcription factor TCF/LEF and CBP to regulate its target genes (Rao et al., Circ. Res., 2010, 106, 1798-1806; The Wnt Homepage http://www.stanford.edu/group/nusselab/cgi-bin/wnt/).

Aberrant upregulation of Wnt signaling is associated with cancer, osteoarthritis, and polycystic kidney disease, while aberrant downregulation of Wnt signaling has been linked to osteoporosis, diabetes, and neuronal degenerative diseases. The Wnt/β-catenin pathway is a therapeutic target for human cancers and various Wnt inhibitors have been examined in preclinical and clinical studies of various human cancers: PORCN inhibitors, Wnt ligand antagonists (FZD decoy receptor), FZD antagonists/monoclonal antibodies, CBP/β-catenin binding inhibitors and β-catenin target inhibitors. Tankyrase inhibitors, including XAV939, JW-55, RK-287107, and G007-LK, which downregulate β-catenin stabilization by stabilizing axin, have also been developed (Review in Jung and Park, Experimental & Molecular Medicine, 2020, 52, 183-191; The Wnt Homepage http://www.stanford.edu/group/nusselab/cgi-bin/wnt/). WO 2009/059994 discloses the tankyrase inhibitor XAV939 and its use for the treatment of Wnt signaling-related disorder associated with aberrant upregulation of Wnt signaling, in particular cancer, osteoarthritis, and polycytic kidney disease.

The cytokines TGF-β are involved in many cellular functions, such as inflammation, cell proliferation and differentiation. The TGF-β pathway is composed of three cytokines of similar structure: TGF-β1, 2 and 3 and transmembrane receptors. It mainly activates the Smad channel, but also the Erk, JNK, p38 MAPK and GTPase channels (Umbarkar et al., JACC Basic Transl. Sci., 2019, 4, 41-53). Aberrant upregulation of TGF-β signaling is associated with cancer and fibrosis. The TGF-β pathway is a therapeutic target for human cancers and fibrotic diseases. TGF-β pathway inhibitors have been examined in preclinical and clinical studies of various human cancers and fibrotic diseases such as idiopathic pulmonary fibrosis; scleroderma, scarring and others: anti-TGFβ2 antisense oligonucleotides (AP-12009, AP-11014, NovaRx); small-molecule TGFβRI or TGFβRI&RII kinase inhibitors (LY-2157299, SB-431542 and many others); anti-pan TGFβ antibody (GC-1008; ID11, SR-2F, 2G7); peptide-fragment of TGFβRIII (P-144); Smad-interacting peptide aptamers (Trx-xFoxH1b/Trx-Lef1); anti-TGFβ2 antibody (Lerdelimumab or CAT-152); anti-TGFβ1 antibody (Metelimumab or CAT-192); stabilized soluble TGFβRII (soluble TBR2-Fc); Review in Akhurst, R J, Current Opinion in Investigational Drugs, 2006, 7, 513-521; Nagaraj N. S. & Datta P. K., Expert Opin. Investig Drugs, 2010, 19, 77-91).

Wnt and TGF-β signaling are also targets for regenerative medicine through differentiation of mammalian pluripotent stem cell population or reprogramming of mammalian differentiated cells. WO 2014/078414 discloses an in vitro method of producing a cardiomyocyte population from a mammalian pluripotent stem cell population by contacting the population successively with a Wnt signaling agonist such as GSK-03 inhibitor (BIO, CHIR-99021) and Wnt signaling antagonist such as C59, IWR-1, IWP-2, IWP-4 and XAV-939. US 2015/0329821 discloses an in vitro method of differentiating stem cells into one or more cell lineages using various combinations of activators or inhibitors of the TGFβ/nodal, Wnt/β-catenin, BMP, PI3K/mTOR, and/or FGF/MAPK signaling pathways.

A combination of Wnt signaling inhibitor (XAV939) and TGF-β signaling inhibitor (SB-431542) has been previously reported to improve reprogramming and cardiac function in vivo in the hearts of mice after surgically-induced myocardial infarction in combination with a cocktail of transcription factors (Gata4, Mef2c and Tbx5; Mohammed et al., Circulation, 2017, 135, 978-995). The effect of the inhibitors was related to the presence of transcription factors, as without them there was no improvement in the surgically-induced model of myocardial infarction.

### SUMMARY OF THE INVENTION

The inventors have found that the Wnt and TGF-β pathways are both impaired and their genes overexpressed in two models of genetically-induced dilated cardiomyopathies, Duchenne muscular dystrophies (DBA2mdx mice) and titinopathies (DeltaMex5 mice). Using the DeltaMex5 mice model which is a severe model of dilated cardiomyopathies, the inventors have shown that a small-molecule inhibitor of the Wnt pathway (XAV939) and a small molecule inhibitor of the TGF-β pathway (SB-431542), in particular in combination, improve the cardiac function. In particular, the small-molecule Wnt and TGF-β pathway inhibitors decrease the cardiac dilatation (reduction of left ventricle diameter, volume, mass) and increase the systolic function (ejection fraction) in the treated DeltaMex5 mice to levels comparable to that of normal mice. These results are surprising in view of previous reports showing that a Wnt signaling inhibitor (XAV939) and a TGF-β signaling inhibitor (SB-431542) did not improve reprogramming and cardiac function in the absence of a cocktail of transcription factors (Gata4, Mef2c and Tbx5) in vivo in a surgically-induced model of myocardial infarction.

These results obtained in two different genetic dilated cardiomyopathies caused by mutations in different genes demonstrate the role of the Wnt and TGF-β pathways in dilated cardiomyopathies, in particular genetic dilated cardiomyopathies and provide a new therapeutic approach for the treatment of these diseases using Wnt and TGF-β inhibitors, in particular in combination.

The use of Wnt and TGF-β pathway inhibitors advantageously provide a functional benefit to the heart as opposed to current treatment of dilated cardiomyopathies which improve the symptoms but do not treat the causative mechanisms of the disease. The use of these inhibitors also offer a therapeutic approach for titinopathies for which gene transfer approaches are not possible because of the size of the gene.

Therefore, the invention relates to a non-expressible inhibitor of the Wnt or TGF-β pathway, or a combination thereof for use in the treatment of dilated cardiomyopathies.

In some embodiments, the inhibitor inhibits the activity of a target protein of the Wnt or TGF-β pathway. The inhibitor is advantageously a small-molecule inhibitor.

In some preferred embodiments, the inhibitor is a small-molecule Wnt inhibitor selected from the group consisting of: Porcupine inhibitors, inhibitors of β-catenin transcriptional activity, Wnt inhibitors, CK1 agonists, LRP6 inhibitors, dishevelled inhibitors and tankyrase inhibitors; preferably a tankyrase inhibitor.

In some more preferred embodiments the small-molecule Wnt inhibitor is selected from the group consisting of: JW-55, FH535, ICG-001 and XAV939; preferably XAV939.

In some preferred embodiments, the inhibitor is a small-molecule TGFβRI or TGFβRI and TGFβRII kinase inhibitor selected from the group consisting of: LY2157299, LY210976, GW788388 and SB-431542; preferably SB-431542.

In some embodiments, the inhibitor inhibits the expression of a target gene of the Wnt or TGF-β pathway. The inhibitor is advantageously an anti-sense oligodeoxyribonucleotide (DNA) or mixed oligonucleotide, preferably targeting the dishevelled, LRP5, LRP6, TGFB1 or TGFB2 gene transcripts, more preferably trabedersen (AP-12009).

In some preferred embodiments, the Wnt pathway inhibitor is used in combination with the TGF-β pathway inhibitor; preferably XAV939 is used in combination with SB-431542.

In some preferred embodiments, the inhibitor is for use in the treatment of genetic cardiomyopathies, preferably caused by mutation(s) in a gene selected from the group consisting of: laminin, emerin, fukutin, fukutin-related protein, desmocollin, plakoglobin, ryanodine receptor 2, sarcoplasmic reticulum Ca(2+) ATPase 2 isoform alpha, phospholamban, lamin A/C, dystrophin, telethonin, actinin, desmin, cardiac actin, sarcoglycans, titin, cardiac troponin, myosin, RNA binding motif protein 20, BCL2-associated athanogene 3, desmoplakin, tafazzin and sodium channels; more preferably the dystrophin or titin gene.

### DETAILED DESCRIPTION OF THE INVENTION

### Inhibitors

The invention relates to the use of a non-expressible inhibitor of the Wnt or TGF-β pathway, or a combination thereof for the treatment of dilated cardiomyopathies (DCM).

As used herein “a non-expressible inhibitor» refers to an inhibitor which cannot be produced by recombinant DNA technologies or delivered by gene transfer. A non-expressible inhibitor is a chemical molecule inhibitor consisting of an organic compound which is different from of a ribonucleotide or amino acid polymer of any length, i.e a ribonucleic acid (RNA) molecule, protein, polypeptide or peptide. An RNA molecule may be partially or totally single-stranded or double-stranded. An RNA molecule includes in particular an interfering RNA (miRNA, siRNA, shRNA), CRISPR guide RNA, ribozyme and aptamer. Any expressible protein, polypeptide or peptide is excluded from the present invention; in particular components of the Wnt or TGF-β pathway, variants or derivative thereof (fragments, fusion proteins; decoy receptors, soluble proteins, dominant negative mutants) and antibodies directed to components of the Wnt or TGF-β pathway including fragments and expressible derivative thereof are excluded from the present invention.

As used herein peptide or polypeptide are used interchangeably to refer to a peptide or protein fragment of any length.

As used herein, the term “muscle cells” refers to myocytes, myotubes, myoblasts, and/or satellite cells.

“a”, “an”, and “the” include plural referents, unless the context clearly indicates otherwise. As such, the term “a” (or “an”), “one or more” or “at least one” can be used interchangeably herein; unless specified otherwise, “or” means “and/or”.

As used herein, “a component of the Wnt or TGF-β (TGF-beta) pathway” refers to any component of this pathway including a ligand, receptor, signaling molecule, or regulator of the Wnt or TGF-β pathway. Such components are well-known in the art (see for example, Rao et al., Circ. Res., 2010, 106, 1798-1806 and Umbarkar et al., JACC Basic Transl. Sci., 2019, 4, 41-53; The Wnt Homepage http://www.stanford.edu/group/nusselab/cgi-bin/wnt/).

An “inhibitor of the Wnt or TGF-β pathway”, “inhibitor of Wnt or TGF-β signaling” or “inhibitor of the Wnt or TGF-β signaling pathway” refers to a compound or molecule which inhibits Wnt or TGF-β signaling, .e.g., the transcription of Wnt or TGF-β target gene(s) through the signaling cascade transduced by the binding of a Wnt ligand or TGF-β cytokine to its cognate receptor. The inhibitor acts on a specific component of the Wnt or TGF-β pathway (Wnt or TGF-β pathway target gene or protein). The inhibitor may inhibit the expression or activity of a positive regulator or activate the expression or activity of a negative regulator of the pathway. The target may be any component of the Wnt or TGF-β pathway such as a ligand, receptor, signaling molecule, or regulator of the Wnt or TGF-β pathway. The inhibition may be direct or indirect. A direct inhibition is directed specifically to the target. An indirect inhibition is directed to any co-effector of the target such as with no limitations: a ligand or co-ligand, a receptor or co-receptor, or a co-factor of said target. The inhibitor may bind to a specific target protein of the Wnt or TGF-β pathway and disrupt specific protein/protein interactions of the target or inhibits the activity or function of the target. Alternatively, the inhibitor may bind to a specific sequence of a target gene transcript (mRNA) and inhibits expression of the target gene of the Wnt or TGF-β pathway.

Typically an inhibitor of the Wnt or TGF-β pathway refers to a compound that inhibits Wnt or TGF-β signaling by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the Wnt or TGF-β signaling prior to or in the absence or, administration of said compound.

Typically an inhibitor of the Wnt or TGF-β pathway may be a compound that inhibits expression or activity of a positive regulator component of the Wnt or TGF-β pathway by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the expression or activity of the component of the Wnt or TGF-β pathway prior to or in the absence or, administration of said compound. As used herein, inhibition of Wnt or TGF-β pathway target gene expression includes any decrease in expression or protein activity or level of the Wnt or TGF-β pathway target gene or protein encoded by said Wnt or TGF-β pathway target gene as compared to a situation wherein no inhibition has been induced. The decrease can be of at least, 20%, 30%, 40%, 50%, 60%, 70%, 80%, 90%, 95%, 99% as compared to the expression of Wnt or TGF-β pathway target gene or level of the Wnt or TGF-β pathway target protein which has not been targeted by inhibition.

An inhibitor of the Wnt or TGF-β pathway may also be a compound that stimulates (or activates) expression or activity of a negative regulator component of the Wnt or TGF-β pathway by at least 20%, 30%, 40%, 50%, 60% and preferably more than 70%, even more preferably more than 80%, more than 90%, more than 95%, more than 99% or even 100% (corresponding to no detectable activity) in a subject (or in a cell in vitro) as compared to the expression or activity of the component of the Wnt or TGF-β pathway prior to or in the absence or, administration of said compound. As used herein, stimulation of Wnt or TGF-β pathway target gene expression includes any increase in expression or protein activity or level of the Wnt or TGF-β pathway target gene or protein encoded by said Wnt or TGF-β pathway target gene as compared to a situation wherein no stimulation has been induced. The increase can be of at least, 20%, 30%, 40%, 50%, 60%, 70%, 80%, 90%, 95%, 99% as compared to the expression of Wnt or TGF-β pathway target gene or level of the Wnt or TGF-β pathway target protein which has not been targeted by stimulation.

In the context of the present invention, an inhibitor of the Wnt or TGF-β pathway according to the present invention is preferably selective for its target protein or gene. By “selective” it is meant that the affinity of the inhibitor is at least 10-fold, preferably 25-fold, more preferably 100-fold, and still preferably 500-fold higher than the affinity for another protein or gene.

The inhibitory activity of a compound on the Wnt or TGF-β pathway may be evaluated by various assays that are well-known in the art. For example chemical libraries are screened in a cellular Wnt or TGF-β reporter assay. Examples of reporter assay include the TOP-flash assay (Molenaar et al., Cell, 1996, 86, 391-399) which is widely used and variants of TOP-flash are available. Another assay is the TCF/LEF-reporter assay using SW480 cells bearing a mutation in the APC protein which leads to constitutively active canonical Wnt signaling (Deshmukh et al., Osteoarthritis and Cartilage, 2018, 26, 18-27).

According to the invention, an inhibitor of the Wnt or TGF-β pathway can be selected among any non-expressible compound having the ability to inhibit activity or gene expression of any positive regulator component of the Wnt or TGF-β pathway or to stimulate activity or gene expression of any negative regulator component of the Wnt or TGF-β pathway.

The Wnt or TGF-β pathway inhibitor or combination thereof according to the invention is used to improve cardiac function and/or fibrosis in subjects suffering from dilated cardiomyopathies (DCM). As shown in the examples of the present application, the Wnt or TGF-β pathway inhibitor or combination thereof is necessary and sufficient to improve cardiac function and/or fibrosis in subjects suffering from dilated cardiomyopathies (DCM); no additional compound such as transcription factors (Gata4, Mef2c and Tbx5) are required to produce the effect. Improvement of cardiac function and/or fibrosis are determined by administration of the Wnt or TGF-β pathway inhibitor or combination thereof in animal models of DCM such as mouse models that are well-known in the art and disclosed in the examples of the present application; DeltaMex5 mice which have the deletion of the penultimate exon (Mex5) of the titin gene (titin^{Mex5-/Mex5-}; Charton et al., Human molecular genetics, 2016, 25, 4518-4532) and DBA2mdx mice which have a punctual mutation on exon 23 of the dystrophin gene. Improvement of cardiac function may be determined by a decrease in cardiac dilatation such as a decrease in left ventricle diameter, volume or mass or an increase in systolic function such as an increase in ejection fraction (EF) after ultrasound analysis in the treated animals compared to untreated controls. Improvement of fibrosis in the heart may be determined by a decrease of fibrotic tissue in the heart after histological analysis (Sirius Red staining) or a decrease of fibrosis marker level (fibronectin, vimentin, collagen 1a1 and collagen 3a1) in the heart after RT-PCR or immunohistological analysis in the treated animals compared to untreated controls. Normal mice (untreated) are advantageously used as positive control to evaluate the efficiency of the treatment in the diseased mice.

In some embodiments, the inhibitor is synthetic.

In some embodiments, the inhibitor inhibits the expression of a target gene of the Wnt or TGF-β pathway. In particular, the inhibitor is a synthetic an anti-sense oligodeoxyribonucleotide (DNA) or mixed oligonucleotide (DNA/RNA) molecule that may be modified. Said synthetic anti-sense oligonucleotide (ASO) which cannot be produced by recombinant DNA technologies or delivered by gene transfer is produced by standard chemical synthesis methods. Said synthetic anti-sense oligonucleotide (ASO) of the invention targets gene transcripts (mRNA) of the Wnt or TGF-β pathway. For example, the ASO may target the dishevelled (DVL), LRP5 or LRP6 gene transcripts(s) of the Wnt pathway or the TGFB1 or TGFB2 gene transcripts of the TGF-β pathway. Examples of ASO targeting the TGFB1 or TGFB2 gene transcripts include trabedersen or AP-12009 (TGFB2 gene), AP-11014 (TGFB1 gene) and NovaRx (TGFB1& TGFB2 genes) which have been examined respectively in Phase III, preclinical and Phase I/II studies of human cancers (Schlingensiepen et al., Cytokine Growth Factor Rev, 2006, 17, 129-139; Schlingensiepen et al. J. Clin. Oncol., 2004, 22, Abs 3132; Review in Akhurst, R J, Current Opinion in Investigational Drugs, 2006, 7, 513-521; Nagaraj N. S. & Datta P. K., Expert Opin. Investig Drugs, 2010, 19, 77-91). The anti-sense oligonucleotide of the invention would act to directly block translation of the target protein and thus preventing protein translation or increasing mRNA degradation, thus decreasing the level of target protein and thus its activity in a cell. For example, antisense oligodeoxyribonucleotides or mixed oligonucleotides of at least about 15 nucleotides and complementary to unique regions of mRNA transcript sequence of a target gene can be synthesized, by known methods. These include techniques for chemical synthesis such as, e.g., by solid phase phosphoramadite chemical synthesis. Methods for using antisense techniques for specifically inhibiting gene expression of genes whose sequence is known are well known in the art (see for example U.S. Pat. Nos. 6,566,135; 6,566,131; 6,365,354; 6,410,323; 6,107,091; 6,046,321; and 5,981,732). The anti-sense oligodeoxyribonucleotide or mixed oligonucleotide (ASO) is usually single-stranded and of less than 100 nucleotides in length and may be modified. Various modifications to the oligonucleotides of the invention can be introduced as a means of increasing intracellular stability and half-life. Possible modifications include but are not limited to the use of phosphorothioate or 2′-0-methyl rather than phosphodiesterase linkages within the oligonucleotide backbone. In some preferred embodiments, the inhibitor is an antisense oligodeoxynucleotide (ASO) delivered into immune cells to prevent TGFβ synthesis such as for example trabedersen (AP-12009).

Anti-sense oligonucleotides of the invention may be delivered in vivo alone or in association with a delivery agent which is different from a gene delivery vector. Agents other than gene delivery vectors which are used to introduce nucleic acid into individual's cells by various means include in particular polymer-based, particle-based, lipid-based, peptide-based delivery vehicles or combinations thereof, such as with no limitations cationic polymer, dendrimer, micelle, liposome, exosome, microparticle and nanoparticle including lipid nanoparticle (LNP); and cell penetrating peptides (CPP). CPP are in particular cationic peptides such as poly-L-Lysine (PLL), oligo-arginine, Tat peptides, Penetratin or Transportan peptides and derivatives thereof such as for example Pip.

In some embodiments, the inhibitor inhibits the activity of a target protein of the Wnt or TGF-β pathway. In particular, the inhibitor is a small organic molecule. The term “small organic molecule” refers to a molecule of a size comparable to those organic molecules generally used in pharmaceuticals. The term excludes biological macro molecules (e. g., proteins, nucleic acids, etc.). Preferred small organic molecules range in size up to about 5000 Da, more preferably up to 2000 Da, and most preferably up to about 1000 Da. The upper molecular-weight of about 900 Da allows for the possibility to rapidly diffuse across cell membranes so that it can reach intracellular sites of action. This molecular weight cutoff is also necessary for oral bioavailability as it allows for transcellular transport through intestinal epithelial cells.

Various small-molecule inhibitors of the Wnt or TGF-β pathway are known in the art and have been examined in clinical trials of various Wnt signaling-associated human cancers (Review in Jung and Park, Experimental & Molecular medicine, 2020, 52, 183-191; Akhurst, R J, Current Opinion in Investigational Drugs, 2006, 7, 513-521; Nagaraj N. S. & Datta P. K., Expert Opin. Investig Drugs, 2010, 19, 77-91; The Wnt Homepage http://www.stanford.edu/group/nusselab/cgi-bin/wnt/) and any of these compounds may be used in the invention. Small-molecule inhibitors are advantageous for the point of view of simplicity and cost of administration.

Porcupine inhibitors block the secretion of WNT ligands through inhibition of post-translational acylation of WNT ligands. Examples of PORCN inhibitors include WNT974, ETC-1922159, RXC004 and CGX1321 which have been examined in Phase 1 or Phase 2 (WNT974) clinical trials of human cancers. Various PORCN inhibitors are disclosed in Dodge et al. (J. Biol. Chem., 2012, 287, 23246-) and Wang et al. (J. Med. Chem., 2013, 56, 2700-). Other PORCN inhibitors include IWP (Chen et al., Nat. Chem. Biol., 2009, 5, 100-107), C59 (Profitt et al., Cancer Res., 2013, 73, 502-), ETC-159 (Madan et al., Oncogne, 2016, 35, 2197-).

Salinomycin, rotlerin and monensin induce the phosphorylation of LRP6, resulting in the degradation of LRP6. Niclosamide promotes FZD1 endocytosis, which down-regulates WNT3A-stimulated β-catenin stabilization. CK1 agonists such as Pyrvinium inhibit the Wnt pathway (Chen et al., Nat. Chem. Biol., 2009, 5, 100-107). Ant1.4Br/Ant1.4C1 is a Wnt inhibitor (Morrell, PLoS One, 2008, 13, 3; e2930.doi:10.371). Apicularen and bafilomycin targets the Vacuolar ATPase (Cruciat et al., Science, 2010, 327, 459-; doi:10.1126). NSC668036 inhibits dishhevelled by binding to its PDZ domain (Shan et al., Biochemistry, 2005, 44, 15495; doi:10.1021)

Wnt/β signaling may be inhibited by restoration of the β-catenin destruction complex. The poly-ADP-ribosylating enzyme tankyrase interacts with and degrades AXIN via ubiquitin-mediated proteosomal degradation. Tankyrase inhibitors which down-regulate β-catenin stabilization by stabilizing AXIN have been developed including XAV939 (CAS 284028-89-3); Huang et al., Nature, 2009, 461, 614-.doi: 10.1038), JW-55, RK-287107, G007-LK; IWR-1 and G244-LM (Chen et al., Nat. Chem. Biol., 2009, 5, 100-107; Kulak et al., Mol. Cell. Biol., 2015, 35, 2425-. doi: 10.1128; Lau et al., Cancer Res., 2013, 73, 3132-. doi: 10.1158/0008); MSC2504877 (Menon et al., Nature Scientific Reports, 2019, 9, 201); RK-287107 (Mizutani et al., Cancer Sci., 2018, 109, 4003-4014); 2X-121 (E7449; Gonigle et al., Oncotarget, 2015, 6, 41307).

Inhibitors of β-catenin transcriptional activity have been developed. PRI-724 inhibits the interaction between CBP and β-catenin and prevents transcription of Wnt target genes and has been examined in Phase 1 clinical trials of human cancers. Carnosic acid, compound 22 and SAH-BLC9 are inhibitors of TCF/LEF and β-catenin interactions which inhibit the formation of the transcriptional complex of β-catenin with coactivators, including BCL9 and PYGO. Pyrvinium downregulates Wnt transcriptional activity through the degradation of PYGO. SM08502 is a small molecule which down-regulates Wnt-signaling-controlled gene expression by inhibiting serine and arginine-rich splicing factor (SRSF) phosphorylation and thereby disrupting spliceosome activity. SM08502 has been examined in Phase 1 clinical trials of human cancers. MSAB (methyl3-[4-methyl)sulfonyl]amino-benzoate) binds to β-catenin and facilitates the ubiquitination-mediated proteosomal degradation of β-catenin. 2,4-diamino-quinazoline inhibits TCF/β-catenin (Chen et al., Nat. Chem. Biol., 2009, 5, 100-107). Quercetin inhibits TCF (Park et al., Biochem. Biophys. Res. Commun., 2005, 328, 227-.doi:10.1016). ICG-001 (CAS No.:780757-88-2) inhibits β-catenin/CREB-binding protein transcription (Emami et al., Proc. Natl. acad. Sci. USA, 2004, 101, 12682-. doi:10.1073). PKF1115-584 and other compounds inhibit TCF/β-catenin complex (Lepourcelet et al. Cancer Cell, 2004, 5, 91-.doi:10.1016). BC2059 inhibits β-catenin/TBL interaction (Fiskus et al., Molecular Cancer Therapeutics, 2011, 10.doi:10.1158; Fiskus et al., Leukemia, 2015, 29, 1267; doi:10.1038).

Other inhibitors of Wnt pathway include SM0469 (CAS number: 1467093-03-03); FH535, iCRT14, KY02111 and CX-4945. (Deshmukh et al., Osteoarthritis and Cartilage, 2018, 26, 18-27); Shizokaol D (Dimeric Sesquiterpene; Tang et al., PloS One, 2016, 11: e0152012.doi:10.1371).

Various ATP mimetic drugs that target the kinase domain of the TGF-β receptor I (TGFβRI; activing receptor-like kinase (ALK)5) or TGF-β receptor I (TGFβRI) & TGF-β receptor II (TGFβRII) and are designed to be specific for Smad2 and Smad3 have been developed. Examples of small-molecule TGFβRI kinase inhibitors include: LY-2157299 (galunisertib), LY-550410, LY-580276, SD-208, SD-093, HTS-466284, SB-505124, SB-431542 (CAS 301836-41-9), Ki-26894, Sm16; NPC-30345, A-83-01, SX-007, IN-1130 that have been examined in preclinical studies of human cancer and LY-573636 that has been tested in Phase II clinical studies of human cancer. Examples of small-molecule TGFβRI & RII kinase inhibitor include: LY-364947 and LY-2109761 (or LY210976) that have been examined in preclinical studies of human cancer (Review in Akhurst, R J, Current Opinion in Investigational Drugs, 2006, 7, 513-521; Nagaraj N. S. & Datta P. K., Expert Opin. Investig Drugs, 2010, 19, 77-91. SB-431542 is an inhibitor of the TGF-β superfamily Type I activin receptor-like kinase (ALK) ALK4, ALK5 (Inman et al., Molecular Pharmacology, 2002, 62, 65-74)). GW788388 is a potent and selective inhibitor of TGFβRI (Gellibert et al., J. Med. Chem., 2006, 49, 2210-2221).

Identification of new small molecule inhibitors can be achieved according to classical techniques in the field. The current prevailing approach to identify hit compounds is through the use of a high throughput screen (HTS). Small molecules agents can be identified from within a small molecule library, which can be obtained from commercial sources such as AMRI (Albany, N.Y.), AsisChem Inc. (Cambridge, Mass.), TimTec (Newark, Del.), among others, or from libraries as known in the art. New small molecule inhibitors may be identified using known reporter assays as disclosed above.

In some preferred embodiments, the Wnt pathway inhibitor is a small-molecule selected from the group consisting of: Porcupine inhibitors, inhibitors of β-catenin transcriptional activity, Wnt inhibitors, CK1 agonists, LRP6 inhibitors, dishevelled inhibitors and tankyrase inhibitors: preferably a tankyrase inhibitor.

In some preferred embodiments, the small-molecule Wnt pathway inhibitor is selected from the group consisting of: JW-55, FH535-, ICG-001 and XAV939; preferably XAV939.

In some preferred embodiments, the TGF-β pathway inhibitor is a small-molecule TGFβRI or TGFβRI & RII kinase inhibitor, preferably selected from the group consisting of: galunisertib [LY2157299], LY210976, GW788388 and SB-431542; more preferably SB-431542.

The invention encompasses the use of one or more Wnt pathway inhibitors or TGF-β pathway inhibitors, alone or in combination. In combination refers to the use of the Wnt pathway inhibitor(s) and TGF-β pathway inhibitor(s) in the same pharmaceutical composition or in different pharmaceutical compositions that are administered simultaneously, separately or sequentially. In some embodiments, the use of the combination produces a much more important effect (cardiac function improvement and/or fibrosis reduction) that the simple addition of the effects that are observed, when the Wnt pathway inhibitor(s) and TGF-β pathway inhibitor(s) are use separately.

In some embodiments, a small-molecule Wnt pathway inhibitor selected from the group consisting of: Porcupine inhibitors, inhibitors of β-catenin transcriptional activity, Wnt inhibitors, CK1 agonists, LRP6 inhibitors, dishevelled inhibitors and tankyrase inhibitors; preferably a tankyrase inhibitor is used in combination with a small-molecule TGF-β pathway inhibitor, preferably a small-molecule TGFβRI or TGFβRI & RII kinase inhibitor, or an antisense TGF-β inhibitor. The combination advantageously comprises a small-molecule Wnt pathway inhibitor selected from the group consisting of: JW-55, FH535, ICG-001 and XAV939 and a TGF-β pathway inhibitor selected from the group consisting of: a small-molecule TGFβRI or TGFβRI & RII kinase inhibitor such as galunisertib (LY2157299), LY210976, GW788388 and SB-431542 and anti-sense oligonucleotide such as trabedersen (AP12-009); preferably XAV939 and SB-431542.

### Pharmaceutical Compositions and Treatment

The Wnt or TGF-β pathway inhibitor, or combination thereof is preferably used in the form of a pharmaceutical composition comprising a therapeutically effective amount of the Wnt or TGF-β pathway inhibitor, or combination thereof.

In the context of the invention, a therapeutically effective amount refers to a dose sufficient for reversing, alleviating or inhibiting the progress of the disorder or condition to which such term applies, or reversing, alleviating or inhibiting the progress of one or more symptoms of the disorder or condition to which such term applies. The term “effective dose” or “effective dosage” is defined as an amount sufficient to achieve, or at least partially achieve, the desired effect.

The effective dose is determined and adjusted depending on factors such as the composition used, the route of administration, the physical characteristics of the individual under consideration such as sex, age and weight, concurrent medication, and other factors, that those skilled in the medical arts will recognize.

In the various embodiments of the present invention, the pharmaceutical composition comprises a pharmaceutically acceptable carrier and/or vehicle.

A “pharmaceutically acceptable carrier” refers to a vehicle that does not produce an adverse, allergic or other untoward reaction when administered to a mammal, especially a human, as appropriate. A pharmaceutically acceptable carrier or excipient refers to a non-toxic solid, semi-solid or liquid filler, diluent, encapsulating material or formulation auxiliary of any type.

Preferably, the pharmaceutical composition contains vehicles, which are pharmaceutically acceptable for a formulation capable of being injected. These may be in particular isotonic, sterile, saline solutions (monosodium or disodium phosphate, sodium, potassium, calcium or magnesium chloride and the like or mixtures of such salts), or dry, especially freeze-dried compositions which upon addition, depending on the case, of sterilized water or physiological saline, permit the constitution of injectable solutions.

The pharmaceutical forms suitable for injectable use include sterile aqueous solutions or suspensions. The solution or suspension may comprise additives which are compatible with viral vectors and do not prevent viral vector particle entry into target cells. In all cases, the form must be sterile and must be fluid to the extent that easy syringe ability exists. It must be stable under the conditions of manufacture and storage and must be preserved against the contaminating action of microorganisms, such as bacteria and fungi. An example of an appropriate solution is a buffer, such as phosphate buffered saline (PBS) or Ringer lactate.

The present application encompasses preparations in the form of kit-of-parts, e.g., preparations containing at least one Wnt pathway inhibitor and at least one TGF-β pathway inhibitor according to the present disclosure, as a combined preparation for simultaneous, separate or sequential use in the treatment of dilated cardiomyopathies. Both active ingredients may be thus formulated into separate compositions or into a unique composition.

The Wnt or TGF-β pathway inhibitor or combination thereof, pharmaceutical composition or kit-of parts according to the present invention are used in the treatment of any dilated cardiomyopathy (DCM).

Dilated cardiomyopathy (DCM or CMD) is characterized by cardiac dilatation and reduced systolic function. DCM is the most frequent form of cardiomyopathy and accounts for more than half of all cardiac transplantations performed in patients between 1 and 10 years of age. Causes of DCMs include in particular genetics, and a variety of toxic, metabolic or infectious agents. Toxic or metabolic agents include in particular alcohol and cocaine abuse and chemotherapeutic agents such as for example doxorubicin and cobalt; Thyroid disease; inflammatory diseases such as sarcoidosis and connective tissue diseases; Tachycardia-induced cardiomyopathy; autoimmune mechanisms; complications of pregnancy; and thiamine deficiency. Infectious agents include in particular Chagas disease due to Trypanosoma cruzi and sequelae of acute viral myocarditis such as for example with Coxsackie B virus and other enteroviruses. A heritable pattern is present in 20 to 30% of cases. Most familial DCM pedigrees show an autosomal dominant pattern of inheritance, usually presenting in the second or third decade of life (summary by Levitas et al., Europ. J. Hum. Genet., 2010, 18: 1160-1165).

In genetically-induced dilated cardiomyopathies, most of the genes involved code for structural elements of cardiomyocytes, including extracellular matrix or Golgi apparatus proteins (laminin, fukutin) involved in cellular adhesion and signaling pathways; desmosome proteins (desmocollin, plakoglobin) involved in cellular junctions; sarcoplasmic reticulum proteins (RyR2, SERCA2a, phospholamban) involved in calcium homeostasis; nuclear envelop proteins (lamin A/C) involved in myocardial structural organisation; cytoskeleton proteins (dystrophin, telethonin, α-actinin, desmin, sarcoglycans) involved in cytoskeleton integrity and muscular strength transmission; and sarcomer proteins (titin, troponin, myosin, actin) involved in generation and transmission of muscular strength.

Mutations in many genes have been found to cause different forms of dilated cardiomyopathy (CMD). These include in particular:
CMD1A, dilated cardiomyopathy-1A (OMIM #115200) caused by heterozygous mutation in the lamin A/C gene (LMNA), (OMIM #150330) on chromosome 1q22; or heterozygous mutation in the laminin alpha 2 (LAMA2 or MEROSIN) gene (OMIM #156225; Marques et al., Neuromuscul. Disord., 2014, doi.org/10.10106/);
CMD1B (OMIM #600884) on 9q13; the gene referred as FDC locus was placed in the interval between D9S153 and D9S152. Friedreich ataxia (OMIM #229300), which is frequently associated with dilated cardiomyopathy, maps to the same region as does also the cAMP-dependent protein kinase (OMIM #176893), which regulates calcium-channel ion conductance in the heart. Tropomodulin (OMIM #190930), which maps to 9q22, was a particularly attractive candidate gene.
CMD1C (OMIM #601493) with or without left ventricular noncompaction, caused by mutation in the lim domain-binding 3, LDB3 (or ZASP) gene (OMIM #605906) on 10q23;
CMD1D (OMIM #601494), caused by mutation in the troponin T2, cardiac (TNNT2) gene (OMIM #191045) on 1q32;
CMD1E (OMIM #601154), caused by mutation in the SCN5A gene (OMIM #600163) on 3p22;
CMD1F: The symbol CMD1F was formerly used for a disorder later found to be the same as desmin-related myopathy or myopathy, myofibrillar (MFM) (OMIM #601419);
CMD1G (OMIM #604145), caused by mutation in the titin (TTN) gene (OMIM #188840) on 2q31;
CMD1H (OMIM #604288) on 2q14-q22;
CMD1I (OMIM #604765), caused by mutation in the desmin (DES) gene (OMIM #125660) on 2q35;
CMD1J (OMIM #605362), caused by mutation in the EYA4 gene (OMIM #603550) on 6q23;
CMD1K (OMIM #605582) on 6q12-q16;
CMD1L (OMIM #606685), caused by mutation in the sarcoglycan delta (SGCD) gene (OMIM #601411) on 5q33;
CMD1M (OMIM #607482), caused by mutation in the CSRP3 gene (OMIM #600824) on 11p15;
CMD1N; (OMIM #607487) caused by mutation in the TITIN-CAP (telethonin or TCAP) gene (OMIM #604488).
CMD10 (OMIM #608569), caused by mutation in the ABCC9 gene (OMIM #601439) on 12p12;
CMD1P (OMIM #609909), caused by mutation in the phospholamban (PLN) gene (OMIM #172405) on 6922;
CMD1Q (OMIM #609915) on 7q22.3-q31.1;
CMD1R (OMIM #613424), caused by mutation in the actin alpha, cardiac muscle (ACTC1) gene (OMIM #102540) on 15q14;
CMD1S (OMIM #613426), caused by mutation in the myosin heavy chain 7, cardiac muscle, beta (MYH7) gene (OMIM #160760) on 14q12;
CMD1U (OMIM #613694), caused by mutation in the PSEN1 gene (OMIM #104311) on 14q24;
CMD1V (OMIM #613697), caused by mutation in the PSEN2 gene (OMIM #600759) on 1q42;
CMD1W (OMIM #611407), caused by mutation in the gene encoding metavinculin (VCL; OMIM #193065) on 10q22;
CMD1X (OMIM #611615), caused by mutation in the gene encoding fukutin (FKTN; OMIM #607440) on 9q31;
CMD1Y (OMIM #611878), caused by mutation in the TPM1 gene (OMIM #191010) on 15q22;
CMD1Z (OMIM #611879), caused by mutation in the troponin C (TNNC1) gene (OMIM #191040) on 3p21;
CMD1AA (OMIM #612158), caused by mutation in the actinin alpha-2 (ACTN2) gene (OMIM #102573) on 1q43;
CMD1BB (OMIM #612877), caused by mutation in the DSG2 gene (OMIM #125671) on 1812;
CMD1CC (OMIM #613122), caused by mutation in the NEXN gene (OMIM #613121) on 1p31;
CMD1DD (OMIM #613172), caused by mutation in the RNA binding motif protein 20 (RBM20) gene (OMIM #613171) on 10q25;
CMD1EE (OMIM #613252), caused by mutation in the myosin heavy chain 6, cardiac muscle, alpha (MYH6) gene (OMIM #160710) on 14q12;
CMD1FF (OMIM #613286), caused by mutation in the troponin I, cardiac (TNN13) gene (OMIM #191044) on 19q13;
CMD1GG (OMIM #613642), caused by mutation in the SDHA gene (OMIM #600857) on5p15;
CMD1HH (OMIM #613881), caused by mutation in the BCL2-associated athanogene 3 (BAG3) gene (OMIM #603883) on 10q26;
CMD1II (OMIM #615184), caused by mutation in the CRYAB gene (OMIM #123590) on 6921;
CMD1JJ (OMIM #615235), caused by mutation in the laminin alpha 4 (LAMA4) gene (OMIM #600133) on 6921;
CMD1KK (OMIM #615248), caused by mutation in the MYPN gene (OMIM #608517) on 10q21;
CMD1LL (OMIM #615373), caused by mutation in the PRDM16 gene (OMIM #605557) on 1p36;
CMD1MM (OMIM #615396), caused by mutation in the MYBPC3 gene (OMIM #600958) on 11p11;
CMD1NN (OMIM #615916), caused by mutation in the RAF1 gene (OMIM #164760) on 3p25;
CMD2A (OMIM #611880), caused by mutation in the troponin I, cardiac (TNNI3) gene on 19q13;
CMD2B (OMIM #614672), caused by mutation in the GATAD1 gene (OMIM #614518) on 7921;
CMD2C (OMIM #618189), caused by mutation in the PPCS gene (OMIM #609853) on 1p134;
CMD3A, a previously designated X-linked form was found to be the same as Barth syndrome (OMIM #302060); and
CMD3B (OMIM #302045), an X-linked form of CMD, caused by mutation in the dystrophin gene (DMD, OMIM #300377).

Desmin-related myopathy or myopathy, myofibrillar (MFM) (OMIM #601419) is a noncommittal term that refers to a group of morphologically homogeneous, but genetically heterogeneous chronic neuromuscular disorders. The morphologic changes in skeletal muscle in MFM result from disintegration of the sarcomeric Z disc and the myofibrils, followed by abnormal ectopic accumulation of multiple proteins involved in the structure of the Z disc, including desmin, alpha-B-crystallin (CRYAB; OMIM #123590), dystrophin (OMIM #300377), and myotilin (TTID; OMIM #604103). Myofibrillar myopathy-1 (MFM1) is caused by heterozygous, homozygous, or compound heterozygous mutation in the desmin gene (DES; OMIM #125660) on chromosome 2q35. Other forms of MFM include MFM2 (OMIM #608810), caused by mutation in the CRYAB gene (OMIM #123590); MFM3 (OMIM #609200) (OMIM #182920), caused by mutation in the MYOT gene (OMIM #604103); MFM4 (OMIM #609452), caused by mutation in the ZASP gene (LDB3; OMIM #605906); MFM5 (OMIM #609524), caused by mutation in the FLNC gene (OMIM #102565); MFM6 (OMIM #612954), caused by mutation in the BAG3 gene (OMIM #603883); MFM7 (OMIM #617114), caused by mutation in the KY gene (OMIM #605739); MFM8 (OMIM #617258), caused by mutation in the PYROXD1 gene OMIM #617220); and MFM9 (OMIM #603689), caused by mutation in the TTN gene (titin; OMIM #188840).

Mutations in other genes have also been found to cause different forms of dilated cardiomyopathy. These include:
desmocollin 2 (DSC2, OMIM #125645) responsible for Arrhythmogenic Right Ventricular Dysplasia 11 (OMIM #610476) and dilated cardiomyopathy (Elliott et al., Circ. Vasc. Genet., 2010, 3, 314-322);
junctiun plakoglobin (JUP or plakoglobin; OMIM #173325) responsible for Arrhythmogenic Right Ventricular Dysplasia 12 (OMIM #611528) and dilated cardiomyopathy (Elliott et al., Circ. Vasc. Genet., 2010, 3, 314-322);
ryanodine receptor 2 (RYR2; OMIM #180902) responsible for Arrhythmogenic Right Ventricular Dysplasia 2 (OMIM #600996) and Ventricular tachycardia, catecholaminergic polymorphic 1 (OMIM #604772) and dilated cardiomyopathy (Zahurul, Circulation, 2007, 116, 1569-1576);
ATPase, Ca(2+)-transporting slow-twitch (ATP2A2; ATP2B, sarcoplasmic reticulum Ca(2+) ATPase 2 isoform alpha (SERCA2a); and
emerin (EMD); fukutin-related protein (FKRP); tafazzin (TAZ); desmoplakin (DSP); and Sodium Channels such as SCN1B, SCN2B, SCN3B, SCN4B, SCN4A, SCN5A and others. In some embodiments, the dilated cardiomyopathy is an acquired dilated cardiomyopathy; for example caused by toxic, metabolic or infectious agents according to the present disclosure. The cause of the dilated cardiomyopathy may also be unknown (idiopathic dilated cardiomyopathy).

In some preferred embodiments, the dilated cardiomyopathy is a genetic dilated cardiomyopathy; preferably caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); lamin A/C (LMNA); dystrophin (DMD); TITIN-CAP or telethonin (TCAP); actinin, in particular actinin alpha-2 (ACTN2); desmin (DES); actin, in particular cardiac actin, actin alpha, cardiac muscle (ACTC1); sarcoglycan, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others.

In some particular embodiments, the genetic dilated cardiomyopathy is caused by mutation(s) in a gene selected from the group consisting of: laminin, in particular laminin alpha 2 (LAMA2) and laminin alpha 4 (LAMA4); emerin (EMD); fukutin (FKTN); fukutin-related protein (FKRP); desmocollin, in particular desmocollin 2 (DSC2); plakoglobin (JUP); ryanodine receptor 2 (RYR2); sarcoplasmic reticulum Ca(2+) ATPase 2 isoform alpha (SERCA2a); phospholamban (PLN); dystrophin (DMD); TITIN-CAP or telethonin (TCAP): actinin, in particular actinin alpha-2 (ACTN2); desmin (DES); actin, in particular cardiac actin, actin alpha, cardiac muscle (ACTC1); sarcoglycan, in particular sarcoglycan delta (SGCD); titin (TTN); troponin, in particular cardiac troponin, troponin T2, cardiac (TNNT2); troponin C (TNNC1) and troponin I, cardiac (TNNI3); myosin, in particular myosin heavy chain 7, cardiac muscle, beta (MYH7) and myosin heavy chain 6, cardiac muscle, alpha (MYH6); RNA binding motif protein 20 (RBM20); BCL2-associated athanogene 3 (BAG3); desmoplakin (DSP); tafazzin (TAZ) and sodium channels such as SCN1B, SCN2B, SCN3B, SCN4B, SCN4A, SCN5A and others. Preferably, dystrophin (DMD) or titin (TTN).

The invention provides also a method for treating a dilated cardiomyopathy according to the present disclosure, comprising: administering to a patient a therapeutically effective amount of the Wnt or TGF-β pathway inhibitor or combination thereof, or of the pharmaceutical composition according to the present disclosure.

The invention relates also to the use of the Wnt or TGF-β pathway inhibitor or combination thereof, or of the pharmaceutical composition according to the present disclosure for the treatment of a dilated cardiomyopathy according to the present disclosure.

The invention provides also the use of the Wnt or TGF-β pathway inhibitor or combination thereof, or of the pharmaceutical composition according to the present disclosure in the manufacture of a medicament for treatment of a dilated cardiomyopathy according to the present disclosure.

The invention provides also a pharmaceutical composition for treatment of a dilated cardiomyopathy according to the present disclosure, comprising a Wnt or TGF-β pathway inhibitor or combination thereof according to the present disclosure as an acive component.

The invention provides also a pharmaceutical composition comprising a Wnt or TGF-β pathway inhibitor or combination thereof according to the present disclosure for treating a dilated cardiomyopathy according to the present disclosure.

As used herein, the term “patient” or “individual” includes human and other mammalian subjects that receive either prophylactic or therapeutic treatment. Preferably, a patient or individual according to the invention is a human.

Treatment”, or “treating” as used herein, is defined as the application or administration of a therapeutic agent or combination of therapeutic agents (e.g., a Wnt pathway inhibitor and/or a TGF-β pathway inhibitor) to a patient, or application or administration of said therapeutic agents to an isolated tissue or cell line from a patient, who has a dilated cardiomyopathy with the purpose to cure, heal, alleviate, relieve, alter, remedy, ameliorate, improve or affect the dilated cardiomyopathy, or any symptom of the dilated cardiomyopathy. In particular, the terms “treat or treatment” refers to reducing or alleviating at least one adverse clinical symptom associated with dilated cardiomyopathy, e.g., cardiac dilatation, in particular left ventricle dilatation and reduced systolic function (e.g., reduced ejection fraction).

The term “treatment” or “treating” is also used herein in the context of administering the therapeutic agents prophylactically.

The pharmaceutical composition of the present invention, is generally administered according to known procedures, at dosages and for periods of time effective to induce a therapeutic effect in the patient. The pharmaceutical composition may be administered by any convenient route, such as in a non-limiting manner by infusion or bolus injection, by absorption through epithelial or mucocutaneous linings (e.g., oral mucosa, rectal and intestinal mucosa, etc.). Administration can be systemic, local, or systemic combined with local; systemic includes parenteral and oral, and local includes local and loco-regional. Systemic administration is preferably parenteral such as subcutaneous (SC), intramuscular (IM), intravascular such as intravenous (IV) or intraarterial; intraperitoneal (IP); intradermal (ID), epidural or else. The parenteral administration is advantageously by injection or perfusion.

The Wnt or TGF-β pathway inhibitor or combination thereof, or of the pharmaceutical composition according to the present disclosure may be used in combination with other therapeutic active agents, wherein the combined use is by simultaneous, separate or sequential administration.

The practice of the present invention will employ, unless otherwise indicated, conventional techniques, which are within the skill of the art. Such techniques are explained fully in the literature.

The invention will now be exemplified with the following examples, which are not limitative, with reference to the attached drawings in which:

### FIGURE LEGENDS

FIG. 1: Functional and histological characterization of the heart after treatment with SB431542 and XAV939 in DeltaMex5 mice

A) Protocol. B) Left ventricle diameter (diastole) and Ejection fraction. C) HPS and Sirius red staining staining of the heart. Scale, 500 μm.

### EXAMPLE 1: IDENTIFICATION OF THE SIGNALING PATHWAYS INVOLVED IN DILATED CARDIOMYOPATHY

### 1. Material and Methods

### Mouse Models

The mice used in this study were male titin^{Mex5-/Mex5-} (DeltaMex5) and DBA/2J-mdx (DBA2mdx) strains, and their respective controls, strains C57BL/6 and DBA/2. DeltaMex5 mice have the deletion of the penultimate exon (Mex5) of the titin gene (titin^{Mex5-/Mex5-}; Charton et al., Human molecular genetics, 2016, 25, 4518-4532). DBA2mdx mice are a model of Duchenne muscular dystrophy due to a point mutation on exon 23 of the dystrophin gene. DBA2mdx mice are on a DBA/J background which has a mutation on the LTBP4 gene, a protein that regulates the activity of the TGF signaling pathway-0.

### Muscle Sampling and Freezing

The muscles of interest are collected, weighed and frozen in liquid nitrogen (samples for molecular biology analysis) or in cooled isopentane (samples for histology), after being placed transversely or longitudinally on a piece of cork coated with gum arabic. The hearts are frozen in diastole before being frozen with a diluted butanedione solution (5 mM) in tyrode. The samples are then stored at −80° C. until use. For the Sirius Red Fibrosis observation protocol on the whole heart, the hearts are included whole in paraffin and stored at room temperature. For the transparency protocols, the sampled hearts are stored whole in 4% para formaldehyde and kept at +4° C.

### RNA Extraction and Quantification

Frozen isopentane muscle is cut into 30 μm thick slices on a cryostat (LEICA CM 3050) at −20° C., separated into eppendorf tubes of about 10-15 slices and stored at −80° C. The TRIzol® method for the extraction of total RNA, based on the solvency properties of nucleic acids in organic solvents, is used. The muscle recovery tubes are resupplied with 0.8 mL of TRIzol® (ThermoFisher) supplemented with glycogen (Roche) at a rate of 0.5 μL/mL of TRIzol®. The tubes are placed in the FastPrep-24 (Millipore) homogenizer for a 20s, 4 m.s. cycle. To recover nucleic acids, after a 5-minute incubation on ice, 0.2 mL of chloroform (Prolabo) is added and mixed with TRIzol®. After a 3-minute incubation at room temperature, the two phases, aqueous and organic, are separated by centrifugation at 12000 g for 15 minutes at 4° C. The aqueous phase, containing the nucleic acids, is removed and placed in a new tube. The RNAs are then precipitated by the addition of 0.5 mL isopropanol (Prolabo) followed by a 10-minute incubation at room temperature and centrifugation for 15 minutes at 12000 g at 4° C. The nucleic acid pellet is washed with 0.5 mL 75% ethanol (Prolabo) and again centrifuged for 10 minutes at 12000 g at 4° C. and then air-dried. The nucleic acids are taken up in 50 μL of nuclease free water, 20 μL are set aside for viral DNA analysis, 30 μL are added to RNAsin (Promega) diluted at 1/50 to preserve the RNAs from degradation. The RNAs are then treated with TURBO Dnase (Ambion) to remove residual DNA. A double Dnase treatment is performed for samples intended for sequencing.

For transcriptome analysis specific to signaling pathways, RT2 Profiler PCR Array (Qiagen) plates are used. The screening plates require the use of a compatible RNA extraction kit, the RNeasy Mini Kit (Qiagen) which extracts the RNA on columns, the kit is used following the supplier's instructions, and the RNAs are then processed by Free DNAse RNAse (Qiagen).

An OD reading is then taken on the ND-8000 spectrophotometer (Nanodrop), from 2 μL of RNA to determine their concentration. RNA is stored at −80° C. and DNA at −20° C.

### Measurement of RNA Quality

In the case of RNAs prepared for sequencing, the quality of the RNAs is measured on the Bioanalyzer 2100 (Agilent) which performs capillary electrophoresis of nucleic acids and then their analysis. The quality is visualized by the retention rate and the concentration of the sample in the form of electrophoregrams. A quality score expressed in RIN (for RNA Integrity Number) is calculated for each sample, on a scale of 0 to 10. The RNA Nano chip (Agilent) is used according to the supplier's instructions. A size marker (RNA 6000 Nano Ladder, Agilent) is passed first, to allow evaluation of RNA size in the samples. A marker is added to each sample, emerging at a defined size. For each sample, 1 μL of RNA is deposited on the chip. On the RNA electrophoregram, the ribosomal RNA peaks are observed: 28S (around 4000 nt), 18S (around 2000 nt) and 5S (around 100 nt). The internal marker emerges at the 25 nt position. The INR is calculated as a function of the height and position of the 18S and 28S peaks, the ratio between the 5S, 18S and 28S peaks, and the signal-to-noise ratio. For RNA-seq, the required quality requires an INR of at least 7.

### RNA Sequencing

RNAseq

The samples used for sequencing are total RNA extracted with TRIzol, treated twice with DNAse and having an INR quality >7. 7. Samples of 2 μg RNA at 100ng/μL were sent for sequencing to Karolinka Institute. The sequencing library used was prepared with the TruSeq Stranded Total RNA Library Prep Kit (Illumina) and sequencing was performed according to the Illumina protocol. The reads are associated using Fastq-pair and aligned to the mouse genome (mm10) using STAR align. The number of reads is proportional to the abundance of corresponding RNAs in the sample. The sequencing platform then provides several files per sample, containing the alignment files in bam format, the list of genes identified with the number of reads for each sample compared and the list of genes accompanied by a normalized numerical count value expressed in fragments per kb per million reads (FPKM).

Analysis

Once the files containing the lists of sequenced transcripts were received, the first step in comparing the samples with each other was to merge the files of the different samples. The goal is to obtain a single table containing, for each transcript identified in the study, its number of reads in each sample. Then, an analysis under the R software was performed with the DESeq2 package: from the number of reads, the samples are normalized, and the differential gene expression for each sample is calculated with respect to its control. The expression difference values (or fold change) are expressed in binary logarithm (log 2.FC), they are associated with their adjusted Pvalue padj. Then, a sorting step was performed to remove: genes containing less than 10 reads under all conditions, genes with no significant padj, genes with a log 2.FC between −0.5 and 0.5 for all conditions. The final table was used to identify genes expressed significantly differentially between the different conditions.

Graphic Representation

The alignment of the reads on the mouse genome (mm10) can be observed by viewing the bam files with the Integrative Genomic Viewer (IGV) software. Different R packages are used for the graphical representation of RNAseq results. For Venn diagrams, the VennDiagram package is used. For Volcano Plots, the ggplot2 package is used. The Ingenuity Pathway Analysis software (IPA, Qiagen) and the gene ontology classification system PANTHER are used to visualize the deregulated signaling pathways in the dataset.

Statistics

In all statistical analyses, the differences are considered significant at P<0.05 (*), moderately significant at P<0.01 (**) and highly significant at P<0.001 (***), with P=probability. Bar graphs are shown as means+SEM standard deviations. The graphs are made using the GraphPad software.

### 2. Results

Total RNAseq (RNAseq) sequencing analysis was performed on heart samples from DeltaMex5 and DBA/2-mdx mice and their controls at early and late age of cardiac involvement. For DeltaMex5 mice, ages of 1 and 4 months were chosen, and for DBA/2-mdx mice, ages of 1 and 6 months. The main aim here was to identify genes present when the pathology is established that would be common to both cardiomyopathy models.

The sequencing was done according to the Illumina protocol. The differential expression of genes for each sample is calculated in relation to its control from their read number (>10). The expression difference values (or fold change) are expressed in binary logarithm (log 2.FC) and are associated with their adjusted Pvalue padj. Genes expressed significantly differentially between different conditions are determined by a log 2.FC>10.51 and a padj<0.05.

The volcano plot of the RNAseq data allows visualization for each condition of the distribution of genes and the extent of gene deregulation in the heart, as well as the extent of gene expression. The list of the 30 most deregulated (overexpressed) genes at 4 months in the heart of the DeltaMex5 model is presented in the Table 1.

TABLE 1
Top 30 most deregulated (overexpressed) genes in
the heart of the DeltaMex5 model at 4 months.

| | | | Average | Average |
|---|---|---|---|---|
| Gene | log2FC | padj | DeltaMex5 | C57BL/6 |
| Spp1 | 6.60 | 5.28E−128 | 2162.74 | 6.65 |
| Gm42793 | 4.82 | 3.66E−46 | 212.55 | 0.00 |
| Cilp | 4.77 | 4.70E−278 | 3357.31 | 109.77 |
| Ltbp2 | 4.74 | 2.97E−174 | 2206.18 | 68.03 |
| Gpnmb | 4.68 | l.57E−97 | 764.57 | 19.98 |
| Sprr1a | 4.33 | l.41E−36 | 222.75 | 1.39 |
| Tnc | 4.28 | 2.99E−33 | 4363.96 | 38.66 |
| Gm6166 | 4.24 | 5.99E−39 | 171.45 | 2.01 |
| 8030451A03Rik | 4.22 | 4.64E−35 | 206.04 | 1.72 |
| D030025P21Rik | 4.02 | 5.44E−36 | 153.86 | 2.87 |
| Timp1 | 3.98 | 5.70E−52 | 625.54 | 24.25 |
| Col12a1 | 3.82 | 2.14E−60 | 989.43 | 50.08 |
| Col8a2 | 3.74 | 3.28E−40 | 247.08 | 10.59 |
| Sfrp2 | 3.62 | 2.08E−55 | 501.17 | 30.20 |
| Thbs4 | 3.61 | 3.79E−121 | 919.77 | 66.85 |
| Ptn | 3.40 | l.25E−35 | 290.91 | 17.60 |
| Postn | 3.35 | 1.65E−21 | 12040.11 | 414.51 |
| Mfap4 | 3.31 | 3.99E−57 | 428.05 | 35.05 |
| Piezo2 | 3.27 | 2.87E−31 | 209.01 | 13.45 |
| Gm26771 | 3.26 | 9.27E−27 | 132.29 | 7.36 |
| Col3a1 | 3.22 | 1.72E−61 | 40685.43 | 3682.59 |
| Col14a1 | 3.20 | 9.47E−116 | 2081.22 | 207.54 |
| Ctss | 3.19 | 3.04E−59 | 1773.00 | 162.81 |
| Trem2 | 3.16 | 2.89E−28 | 259.21 | 17.93 |
| Atp6v0d2 | 3.15 | 2.67E−17 | 58.07 | 0.67 |
| Apol7d | 3.15 | 5.17E−32 | 541.35 | 41.67 |
| AC125167.1 | 3.12 | 3.11E−46 | 1533.67 | 141.84 |
| Lgals3 | 3.10 | 6.97E−19 | 747.38 | 35.14 |
| Mpeg1 | 3.09 | 8.80E−23 | 2205.83 | 143.04 |
| Dkk3 | 3.01 | 2.78E−32 | 299.63 | 27.12 |

Underlined = model specific

For the DBA/2-mdx model, the list of the 30 most deregulated (overexpressed) genes in the heart at 6 months is presented in the Table 2.

TABLE 2
Top 30 most deregulated (overexpressed) genes in
the heart of the DBA/2-mdx model at 6 months.

| | | | Average | Average |
|---|---|---|---|---|
| Gene | log2FC | padj | DBA/2-mdx | DBA/2 |
| Ighg2c | 3.99 | 2.34E−40 | 127.71 | 0.00 |
| Tnc | 3.76 | 1.89E−111 | 1637.90 | 96.86 |
| Cilp | 3.27 | 4.59E−70 | 760.53 | 62.18 |
| Sprr1a | 3.02 | 7.35E−22 | 75.33 | 1.37 |
| Mt2 | 2.98 | 3.69E−44 | 425.98 | 39.53 |
| Timp1 | 2.83 | 1.53E−19 | 735.88 | 34.09 |
| 8030451A03Rik | 2.65 | 5.15E−18 | 77.45 | 4.44 |
| Serpina3n | 2.60 | 9.78E−18 | 2728.99 | 200.98 |
| Chile1 | 2.54 | 1.01E−18 | 173.35 | 15.73 |
| Hamp2 | −2.51 | 2.58E−38 | 61.43 | 427.96 |
| Lrp8 | 2.47 | 1.81E−16 | 132.79 | 11.37 |
| Saa3 | 2.41 | 6.08E−13 | 46.74 | 0.65 |
| Fam46b | 2.36 | 5.00E−34 | 511.91 | 83.18 |
| Per2 | 2.35 | 8.14E−32 | 292.28 | 47.22 |
| Fgl2 | 2.34 | 4.46E−65 | 3216.00 | 585.39 |
| Lox | 2.32 | 7.38E−52 | 919.81 | 166.22 |
| Crlf1 | 2.30 | 1.98E−19 | 172.27 | 24.17 |
| Postn | 2.28 | 6.18E−21 | 9086.68 | 1378.81 |
| Ereg | 2.27 | 3.18E−12 | 58.87 | 3.76 |
| Cfb | 2.27 | 4.86E−41 | 632.26 | 115.30 |
| Nxpe5 | 2.27 | 4.06E−28 | 215.52 | 36.33 |
| Gm20547 | 2.27 | 6.53E−48 | 712.93 | 133.02 |
| Ccl6 | 2.26 | l.05E−64 | 1020.70 | 197.87 |
| Ccl9 | 2.24 | 4.79E−43 | 492.52 | 93.30 |
| Pak3 | 2.20 | 3.81E−15 | 117.72 | 15.86 |
| Mmp3 | 2.17 | 7.28E−35 | 967.23 | 187.88 |
| Srpx | 2.17 | 9.38E−31 | 366.18 | 70.08 |
| Clec4d | 2.16 | 1.12E−12 | 66.61 | 7.53 |
| Ccl7 | 2.16 | 2.68E−18 | 140.95 | 22.98 |
| He33 | 2.15 | 2.60E−32 | 353.56 | 69.27 |

Underlined = model specific.

The Top 30 most increased genes in the heart of the DeltaMex5 model at 4 months include genes of the WNT and TGF-β signaling pathways. The Osteopontin gene (Spp1) (log 2FC=6.6, P=5.28E-128) is a target of of the WNT signaling pathway in the heart (Zahradka et al., Circulation Research, 2008, 102, 270-272; Marchand et al., Cell, 2011, 10, 220-232). Two other genes are also found in this list belong directly to the WNT signaling pathway: Sfrp2, coding for Secreted Frizzled-Related Protein-2 (log 2FC=3.62, P=2.08E-55) and Dkk3 coding for Dickkopf related protein-3 (log 2FC=3.01, P=2.78E-32). The third gene is the Cilp gene, coding for Cartilage Intermediate Layer Protein (log 2FC=4.77, P=4.70E-278), a negative regulator of the TGF-β pathway (Shindo et al. Int. Journal of Gerontology, 2017, 11, 67-74). The fourth gene, Ltbp2 (log 2FC=4.74, P=2.97E-174), is a modulator of the TGF-β pathway and codes for the Latent-Transforming growth factor Beta-binding Protein 2 (Sinha et al., Cardiovascular Research, 2002, 53, 971-983). Periostine encoded by Postn (log 2FC=3.351948, P=1.7E-21) and Tissue Inhibitor of Metalloproteinase-1 encoded by Timp1 (log 2FC=3.981384, P=5.7E-52) found in the list of most increased genes in the heart are targets of the TGF-β pathway (Snider et al., Circulation Research, 2009, 105, 934-947; Li et al., Cardiovascular Research, 2000, 46, 214-224). At 1 month, the number of deregulated genes is much smaller and the deregulated genes are deregulated to a lesser extent with a maximum log 2FC of 1.

The Venn diagram representation of RNAseq results allows the visualization of the numbers of common or specific deregulated genes in a model or a stage of disease progression. Of the 46,717 genes included in the RNAseq analysis, 4,850 genes were found to be significantly deregulated (|log 2FC|>0.5 and pvalue<0.05) in either model at early or late age of cardiac involvement compared to control. At an early age, the heart of DeltaMex5 mice has only 44 deregulated genes, whereas the heart of DBA/2-mdx mice already has 2,186, with only 4 genes in common in both models. At a later age, the DeltaMex5 heart has 2,621 deregulated genes and the DBA/2-mdx heart has 2,202, of which 1,175 are common to both models, of which 708 genes are specific for the advanced age of cardiomyopathy. Only 9 genes are specific for the DeltaMex5 model, while 232 are specific for the DBA/2-mdx model. Of all the deregulated genes, a greater proportion of the genes are over-expressed rather than under-expressed. The majority of the most over-expressed genes are common between the two models. However, genes deregulated in the hearts of DeltaMex5 mice at 4 months are more strongly deregulated than genes deregulated in the hearts of DBA/2-mdx mice at 6 months (log 2FC maximum of 4 versus 6.6). It was also observed that, although the cardiac involvement between the two models was different, the transcriptional deregulations associated with them mostly involved the same genes and signaling pathways at a late stage.

To complete this analysis, the Ingenuity Pathway Analysis (IPA, Qiagen) software, which uses a repository of biological interactions and functional annotations to help interpret the data into biological mechanisms was used. At one month of age, no increase in signaling pathways was identified in the hearts of DeltaMex5 and DBA/2-mdx mice. Analysis by IPA allowed to highlight the biological functions whose genes are most represented in the deregulated genes in an advanced phase. In first position in both models, more than 150 genes involved in cardiovascular disease were found in the RNAseq analysis. In second position, more than 150 deregulated genes are categorized in the family of lesions and abnormalities on an organ. Finally, in third position, nearly 200 genes related to the function and development of the cardiovascular system were found.

The inventors also used another function of the IPA software to determine the toxicity associated with the observed changes in gene expression, and this only in the advanced phases. Many deregulated genes were identified: 86 genes associated with cardiac enlargement in the DeltaMex5 model and 85 in the DBA/2-mdx model, 45/48 genes that could lead to cardiac dysfunction, 38/36 genes in cardiac dilatation, 27/28 genes in cardiac fibrosis and 35/37 in cardiac necrosis.

The PANTHER gene ontology classification system was also used to determine the most deregulated signalling pathways in the late-stage models. In both models, the perturbations appear to be very similar as seen in the analysis of the Venn Diagrams. In the late-stage model, the WNT signaling pathway is found in 4^{th} position of the most deregulated signaling pathways in the heart of DeltaMex5 mice and in 3^{rd} position in the heart of DBA/2-mdx mice. A total of 40 genes belonging to this pathway are deregulated (overexpressed), including Sfrp2 and Dkk3. The TGF-β pathway, is found in 22^{nd} and 19^{th} position in the heart of DeltaMex5 and DBA/2-mdx, with more than 15 deregulated genes, to which Cilp and Ltbp2, two of the most over-expressed genes, belong.

The RNAseq analysis shows that the Wnt and TGF-β pathways are both impaired and their genes overexpressed in two models of genetically-induced dilated cardiomyopathies, Duchenne muscular dystrophies (DBA2mdx mice) and titinopathies (DeltaMex5 mice). These results prompted the inventors to assess the effect of the inhibition of these pathways on the cardiac phenotype of the model.

### EXAMPLE 2: TREATMENT OF DILATED CARDIOMYOPATHY MOUSE MODEL WITH WNT AND TGF-β PATHWAY INHIBITORS

### 1. Material and Methods

### Mice Treatment

DeltaMex5 males, 1 month-old were treated by intraperitoneal injection of SB-431542 (10 mg/kg) (Sigma-Aldrich); XAV939 (2.5 mg/kg) (Sigma-Aldrich); SB431542 (10 mg/kg) and XAV939 (2.5 mg/kg), 3 times a week for 3 months. Untreated C57BL/6 and DeltaMex5 mice of same age and sex were used as controls.

### Ultrasound Analysis of Cardiac Function

The mice are anaesthetized by inhalation of isoflurane and placed on a heating platform (VisualSonics). Temperature and heart rate are continuously monitored. The image is taken by a Vevo 770 high-frequency echocardiograph (VisualSonics) with 707B probe. Ultrasound measurements in 2D mode and M mode (motion) are taken along the large and small parasternal axis at the widest level of the left ventricle. Quantitative and qualitative measurements are performed using the Vevo 770 software. The mass of the left ventricle is estimated using the following formula:

Mass of the left ventricle (g)=0.85(1.04(((diameter of the left ventricle at the end of diastole+thickness of the intraventricular septum at the end of diastole+thickness of the posterior wall at the end of diastole)³−diameter of the ventricle at the end of diastole3)))+0.6.

For each ultrasound of a mouse heart, about 5 measurement points are taken. The measuring point corresponding to the maximum size of the left ventricle in diastole is then used, as it represents the maximum dilatation that the mouse heart can reach.

### Histology

Frozen isopentane muscle is cut into 8 μm thick slices on a cryostat (LEICA CM 3050) at −20° C. The slices are placed on a blade and stored at −80° C.

Haematoxylin-Phloxine-Safran Staining

The Hematoxylin-Phloxine-Safran (HPS) marking allows the general appearance of the muscle to be observed and the different tissue and cell structures to be highlighted. Haematoxylin colours nucleic acids dark blue, phloxine colours the cytoplasm pink, saffron colours collagen red-orange.

Cross sections are stained with Harris hematoxylin (Sigma) for 5 min. After washing with water for 2 min, the slides are immersed in a 0.2% (v/v) hydrochloric alcohol solution for 10 s to remove excess stain. After being washed again with water for 1 min, the tissues are blued in a Scott water bath (0.5 g/l sodium bicarbonate and 20 g/l magnesium sulphate solution) for 1 min before being rinsed again with water for 1 min and stained with phloxine 1% (w/v) (Sigma) for 30 s. After rinsing with water for 1 min 30 s, the cuts are dehydrated with 700 ethanol for 1 min and then rinsed in absolute ethanol for 30 s. The tissues are then stained with saffron 1% (v/v in absolute ethanol) for 3 min and rinsed in absolute ethanol. Finally, the cuts are thinned in a Xylene bath for 2 min and then mounted with a slide in the Eukitt medium. Image acquisition is performed with objective 10 on a Zeiss AxioScan white light microscope coupled to a computer and a motorized stage.

From HPS coloured sections, the centronucleation index is calculated by the ratio of the number of centronucleated fibres to the area of the section in mm2.

Sirius Red Coloration

This staining allows the collagen fibres to be coloured red and to highlight the presence of fibrotic tissue. Cytoplasms are stained yellow.

Cross sections are dehydrated with acetone for 1 hour for frozen cuts or dewaxed with heat and toluene baths. They are then fixed with 4% formaldehyde for 5 min then 10 min in a Bouin solution. After two washes with water, the slides are immersed in Sirius Red solution (0.1 g Sirius Red per 100 mL picric acid solution) for 1 h for staining. After rinsing with water for 1 min 30 s, the slices are dehydrated in successive ethanol baths: 700 ethanol for 1 min, 950 ethanol for 1 min, absolute ethanol for 1 min and then a second absolute ethanol bath for 2 min. Finally, the slices are thinned in two Xylene baths for 1 min and then mounted with a lamella in the Eukitt medium. Image acquisition is carried out with objective 10 on a Zeiss AxioScan white light microscope coupled to a computer and a motorized stage.

The polarized light images were acquired using a modified right LEICA microscope with a polarizer placed before the sample (Polarizer), along the path of the light; and another polarizer placed after the sample (Anlayzer), which can be rotated by hand, giving the possibility to observe both transmitted and polarized light at the same time. The main axes of the polarizers are oriented at 90 degrees to each other. Polarized light maps have been acquired using a Retiga 2000 CCD sensor (QImaging) coupled to the Cartograph software (Microvision, France). In summary, the light passes through the first polarizer before reaching the sample, the collagen being birefringent the light that passes through it is separated into two rays, which once passed through the second polarizer will allow the differential observation of the two types of Sirius Red and the rest of the cardiac tissue.

Quantification of Sirius Red

### Sirius Quant

Sirius Quant is an internally developed ImageJ pluggin (Schneider et al., 2012). It is a thresholding macro that allows to isolate and quantify the pixels of the image that are colored red. It works in 3 steps: the first one is to convert the image to black and white. The images resulting from the Red Sirius colorations are very contrasted, so a simple black and white conversion is enough to keep all the useful information. The second one is a very rough thresholding in order to keep only the colored pixels of the image, in other words the pixels belonging to the whole cut. Using the Analyze Particles function with an adapted object size allows automatic detection of the outline of the slice, which is then stored. The third step is a manual thresholding by the user which allows to keep only the pixels colored in red, those associated with the marking. A manual correction tool makes it possible, either to remove areas that would have been detected and that are not marking (dust, cut fold, etc.), or to add areas that would not have been taken into account. Once the thresholded image is satisfactory, the number of thresholded pixels and the total number of pixels in the entire section are then measured. A ratio between these two numbers finally gives the fibrosis index in the slice.

### WEKA

The images were processed using an artificial intelligence algorithm via the WEKA plugin (ImageJ). The WEKA classifier pluggin was implemented using a training data set containing 17 images representative of the different conditions to be classified. The classes were assigned to healthy tissue (yellow), to both types of staining and to slice rupture (white). The original mappings are mosaic images with a size of approximately 225 megapixels (15 k×15 k), which were divided into 400 frames (20 rows, 20 columns), each frame measuring approximately 750×750 pixels. Each frame is classified independently and the complete image is then reconstructed. The number of pixels in each class is measured. The total number of pixels belonging to the heart is calculated as the sum of the healthy tissue and the two types of dye uptake. The ratio of each class is then calculated by dividing the number of pixels in the class by the total number of pixels in the heart.

### Whole-Heart Reconstruction and Quantification

Sections of a whole heart colored by Sirius Red were scanned with a scanner (Axioscan ZI, Zeiss) with a 10× lens. A total of 483 images were obtained. They were aligned using ImageJ's pluggin: Linear Stack Alignment with SIFT (Lowe et al., International Journal of Computer Vision, 2004, 60, 91-110). Some images were manually aligned when the software did not allow a satisfactory alignment. The image was loaded into Imaris (BitPlane, USA) for reconstruction and 3D visualization. Once the images were aligned, the Sirius Quant pluggin in fully automatic mode using Otsu thresholding (Otsu N, Cybernetics, 1979, 9, 62-66) resulted in 483 fibrosis ratio values corresponding to each image. These values were filtered using the sliding average method, which is a method of reducing noise in a signal to avoid the errors inherent in automating an algorithm. The use of the moving average allows to limit these errors by replacing each fibrosis ratio of an image by the average of itself, the ratio of the image preceding it and the ratio of the image following it.

### Statistics

In all statistical analyses, the differences are considered significant at P<0.05 (*), moderately significant at P<0.01 (**) and highly significant at P<0.001 (***), with P=probability. Bar graphs are shown as means+SEM standard deviations. The graphs are made using the GraphPad software.

Analysis of the distribution of fibrosis over the whole heart: In order to ensure that the fibrosis is homogeneous in the heart (HO hypothesis), we randomly drew 20 values from the 483 fibrosis ratio values. These values were compared 10 to 10 with a Wilcoxon test (Software R) to obtain a p-value. This operation was repeated 1000 times, resulting in 1000 p-values. Among these values some are below 0.05 showing that in some cases our hypothesis of fibrosis invariance is not valid. Out of the 1000 statistical tests, we counted how many gave a value below 0.05. We repeated the entire process 100 times to obtain an average of the percentage for which our HO hypothesis is false. This average is 4%. This means that our hypothesis is valid 96% of the time, and therefore corresponds to an overall p-value of 0.04, which is statistically acceptable.

Ultrasound analysis: In order to determine the relationships between the parameters and which parameters are of interest for the study, the statistical software R is used. The scatterplotMatrix function was used to visualize the correlations between the measurements at different ages and to select the parameters to be studied. The statistical analyses are performed with Rcmdr and the graphs with the GraphPad software.

### 2. Results

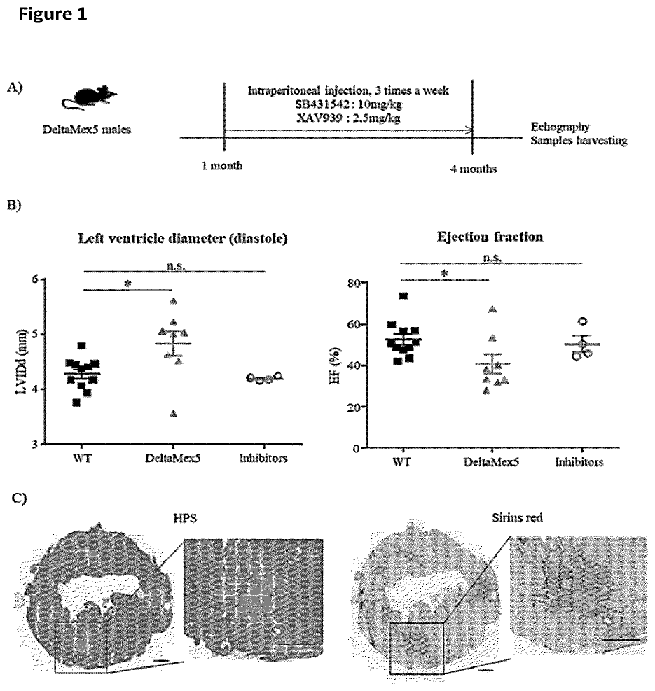

Inhibition of WNT and TGF-β pathways identified by RNAseq as highly dysregulated in the DeltaMex5 model compared to WT was performed by the use of the TGF-β inhibitor SB431542 and the WNT inhibitor XAV939, alone or in combination. Treatment consists in intraperitoneal injection of SB431542 (10 mg/kg) and XAV939 (2.5 mg/kg), 3 times a week for 3 months (FIG. 1A). Injection was initiated at 1 month of age, before the onset of heart impairment, and was ended at 4 months after echography measurements. The consequences on the heart at the global, histological and functional level were studied.

### Morphological Evaluation

Mouse mass was significantly decreased in mice treated with the combination of SB431542 and XAV939 after 3 months (27.93±1.27 g, n=4, versus 34.9±1.3 g, n=8, P<0.01) and returned to a normal value as shown by the lack of difference with C57BL/6 control mice (28.81±0.72, n=11, P>0.05). The ratio of heart mass to total mouse mass was not significantly different between the three groups, but there was still a clear tendency for the proportion of heart to decrease in the C57BL/6 control mice (0.57±0.02%, n=7) and injected DeltaMex5 mice (0.55±0.04%, n=4) compared to untreated DeltaMex5 mice (0.63±0.02%, n=8).

Heart tissue sections of treated mice were stained by HPS and Sirius red to evaluate histological impact of inhibitors treatment. Large areas of damaged tissue are still found in HPS stained tissue and Sirius red staining demonstrate the presence of fibrotic tissue in the whole heart (FIG. 1C). Quantification of total tissue fibrosis by Sirius Red collagen staining shows that the fibrosis rate does not decrease in treated DeltaMex5 mice compared to DeltaMex5 mice (21.87±2.48% versus 18.68±1.74%, P>0.05, n=4).

### Functional Evaluation

The echocardiography measured in mice after inhibitors treatment shows an enhanced cardiac condition as the two mains parameters, LV function and LV dilatation, were improved (FIG. 1B). The LV diameter of treated mice was decreased by 0.63 mm compare to untreated DeltaMex5 (4.20±0.02 mm versus 4.83±0.22 mm, n=4 and 8) and was not different from WT mice (4.28±0.09 mm, n=11). Other related parameters, body mass, LV mass, LV volume, were improved as well. Left ventricular diastole volume was decreased by more than 30 μL (78.47±0.774 μL, n=4 versus 111.4±10.67 μL, n=8) and no longer different from C57BL/6 mice (82.66±3.923, n=11, P>0.05). The same parameters in systole were also improved. Heart hypertrophy was also reduced, resulting in a 40% decrease in left ventricular mass (131.6±14.52 mg, n=4, versus 190±12.77 mg, n=8, P<0.05). Finally, cardiac muscle function is also improved by treatment. Indeed, the ejection fraction of treated mice increase by 9% compare to untreated DeltaMex5 (50±4% versus 41±5%, n=4 and 8) and was not significantly different anymore from the untreated WT mice (53±3%, n=11). In conclusion, the cardiac condition of the mice was improved in left ventricular function and dilatation, although fibrosis of the heart was not decreased.

### Molecular Evaluation

In order to determine the efficacy of the inhibitors on the TGF-β and WNT pathways, the inventors have used an RT2-profiler array (RT2-PCR) screening kit of a set of 84 target genes for the WNT and TGF-β pathways on samples from treated mice. The deregulation of both signaling pathways was verified in the DeltaMex5 model at the age of 4 months. Among the WNT pathway targets included in the test, 27 genes were overexpressed, and 23 genes were overexpressed among the TGF-β pathway targets. Most of the target genes of the WNT and TGF-β pathways were decreased in mice treated with SB431542 and XAV939.

### CONCLUSION

TGF-β inhibitor SB431542 and WNT inhibitor XAV939 concomitant use improved the cardiac function but did not improve tissue fibrosis after 3 months of treatment.

1-15. (canceled)

16. A method of treating cardiomyopathies in a patient in need thereof, comprising administering to the patient a therapeutically effective amount of a non-expressible inhibitor of the Wnt or TGF-β pathway, or a combination thereof.

17. The method according to claim 16, wherein the inhibitor inhibits the activity of a target protein of the Wnt or TGF-β pathway.

18. The method according to claim 16, wherein the inhibitor is a small-molecule inhibitor.

19. The method according to claim 16, wherein the inhibitor is a small-molecule Wnt inhibitor selected from the group consisting of: Porcupine inhibitors, inhibitors of β-catenin transcriptional activity, Wnt inhibitors, CK1 agonists, LRP6 inhibitors, dishevelled inhibitors and tankyrase inhibitors.

20. The method according to claim 19, wherein the small-molecule Wnt inhibitor inhibitor is a tankyrase inhibitor.

21. The method according to claim 19, wherein the inhibitor is a small-molecule Wnt inhibitor selected from the group consisting of: JW-55, FH535, ICG-001 and XAV939.

22. The method according to claim 19, wherein the small-molecule Wnt inhibitor is XAV939.

23. The method according to claim 16, wherein the inhibitor is a small-molecule TGFβRI or TGFβRI and TGFβRII kinase inhibitor.

24. The method according to claim 23, wherein the small-molecule inhibitor is selected from the group consisting of: LY2157299, LY210976, GW788388 and SB-431542.

25. The method according to claim 23, wherein the small-molecule inhibitor is SB-431542.

26. The method according to claim 16, wherein the inhibitor inhibits the expression of a target gene of the Wnt or TGF-β pathway.

27. The method according to claim 16, wherein the inhibitor is an anti-sense oligodeoxyribonucleotide or mixed oligonucleotide.

28. The method according to claim 27, wherein the anti-sense oligodeoxyribonucleotide or mixed oligonucleotide targets the dishevelled, LRP5, LRP6, TGFB1 or TGFB2 gene transcripts.

29. The method according to claim 27, wherein the anti-sense oligodeoxyribonucleotide or mixed oligonucleotide is trabedersen.

30. The method according to claim 16, wherein the Wnt pathway inhibitor is used in combination with the TGF-β pathway inhibitor.

31. The method according to claim 30, wherein XAV939 is used in combination with SB-431542.

32. The method according to claim 16, wherein the cardiomyopathies are genetic cardiomyopathies.

33. The method according to claim 32, wherein the genetic cardiomyopathy is caused by mutation in a gene selected from the group consisting of: laminin, emerin, fukutin, fukutin-related protein, desmocollin, plakoglobin, ryanodine receptor 2, sarcoplasmic reticulum Ca(2+) ATPase 2 isoform alpha, phospholamban, lamin A/C, dystrophin, telethonin, actinin, desmin, cardiac actin, sarcoglycan, titin, cardiac troponin, myosin, RNA binding motif protein 20, BCL2-associated athanogene 3, desmoplakin, tafazzin and sodium channels.

34. The method according to claim 32, wherein the genetic cardiomyopathy is caused by mutation in the dystrophin or titin gene.
